(19) United States
(12) Patent Application Publication
(10) Pub. No.: US 2010/0010565 A1
(43) Pub. Date: Jan. 14, 2010

(54) Extended range wireless muscular and neural stimulation

(76) Inventors: Samuel Victor Lichtenstein, Vancouver (CA); Daniel Gelbart, Vancouver (CA)

Correspondence Address: DANIEL GELBART, 4706 DRUMMOND DR, VANCOUVER, BC, V6T-1B4, CA

(21) Appl. No.: 12/216,648

(22) Filed: Jul. 9, 2008

(51) Int. Cl. A61B 1/00

(52) U.S. Cl. 607/46; 607/61

(57) ABSTRACT

Miniature implanted muscle, nerve and brain stimulators are powered by inductive coupling to a large coil which is preferably placed under the bed. Preferably the operation of the system is controlled by a programmable timer to operate when the user is resting or asleep. Two coils operated at two different positions can be used simultaneously to avoid spots with no signal. When the system is used to reduce angina pains, the pulsation is synchronized to the cardiac rhythm by picking up the electro-cardiac signals.

### FIELD OF THE INVENTION

The invention relates to the medical field and more specifically to electronic muscle and neural stimulation.

### BACKGROUND OF THE INVENTION

It is well known that human muscles and nerves can be stimulated by using electrical pulses. For muscles results closely match those from voluntary muscular activity. For nerves many unexpected benefits, mainly in pain reduction and healing, were found. The stimulation can be done by at least five different methods: by attaching external electrodes, by implanting internal electrodes, by wireless internal electrodes, by electrodes using an internal power source or by inducing a voltage inside the body without the use of electrodes. While the last method seems the most attractive it is not practical for many applications as it requires magnetic fields of about 1 Tesla. Such fields are difficult to achieve without close proximity to a large coil. By comparison, wireless internal electrodes can be made to work with fields under 0.001 Tesla. Stimulators using an internal battery require battery replacement surgery and take up significant space. This is justified for some applications, such as pacemakers, but not for less life-threatening situations. Methods requiring wires, either connected to internal electrodes or external electrodes, are less convenient. One object of the present invention is to exercise the muscles and stimulate nerves while a person is sleeping. In order to achieve stimulation having minimal interference with normal sleep, no stimulation wires should be connected to the body. Prior art wireless devices require a bulky coil attached to the body or placed in close proximity with the implanted electrodes. Such a coil interferes with the natural changes in body position during sleep. Another object of the invention is to provide a fully automatic and non-obtrusive system, preferable starting up gradually while the user is asleep and providing long durations of muscle or neural stimulation. The significance of stimulation while asleep is that many applications require long hours of stimulation which may interfere with activities during waking hours. A well known device in the field of wireless muscle stimulation is the BION™ implant described in U.S. Pat. No. 5,312,439. The BION is a miniaturized implantable electrode complete with a pick-up coil and pulse generator. The small size of the BION, about 2 mm in diameter, allows delivery via a hypodermic needle without surgery. The device can be active during the delivery procedure for optimized placements. By modifying the BION design slightly and by using a different design for the transmitter it is possible to achieve wireless operation over significantly larger distances than the BION. This allows placing the transmitting coil in a more convenient location such as under the bed, and having good coupling to the implanted device regardless of body position on the bed. While these devices are often referred to as having a “transmitter” and a “receiver” coil, they actually operate as an air-coupled transformer, as the receiver is typically in the “near field” part of the electromagnetic field set up by the transmitter. Another term used to describe the coupling between the transmitter coil and the receiver coil is “inductive coupling”. An alternative embodiment is disclosed that operates as a true transmitter and receiver, i.e. the receiver is in the far field of the transmitter and does not rely on transformer action for coupling. The terms “near field” and “far field” are well known in the art of electromagnetic fields. Also the term “muscle stimulation” is used interchangeably with the term “nerve stimulator”. A muscle can be stimulated directly or by stimulating the nerves that normally stimulate the muscle. By finding the correct spot in the nerve bundle a significantly lower energy is required to stimulate the muscle, as the electrical signal stimulates the nerve and the latter stimulates the muscle. In some applications there is no need to stimulate the muscle at all. It has been found that certain illnesses such as depression, epilepsy, migraines and other may respond to electrical neural stimulation in the brain. In such cases there is no muscle stimulation. For such applications the term “muscle stimulation” in this disclosure should be understood to mean “neural stimulation”. In general, the advantage of the proposed system over an implanted power source, such as a pacemaker, is the ability to deliver the implanted part without surgery and avoiding the need for repeat surgery to replace batteries. The disadvantage compared to an implanted power source is that stimulation can only be used when in proximity to the transmitter coil. Fortunately this is not a problem for the application listed and many others, as the stimulation does not need to be continuous and sometimes there are advantages to rest periods between stimulation periods. Being able to place the transmitter coil away from the body, for example, under the bed, opens up applications such as:
building up muscle strength and losing weight while asleep.
helping handicapped and bed ridden patients retain muscle strength.
relieving angina by using leg muscles for counter pulsation of blood to assist the heart.
pelvic floor stimulation to control female incontinence.
control of sleep apnea.
muscle movement for patients who lost the natural ability to move certain muscles.
brain stimulation to control depression.
brain stimulation to control epilepsy.
brain stimulation to control migraine headaches and other pain.
brain stimulation to control alcoholism.
brain stimulation to control back diabetic neuropathic pain.
brain and muscle stimulation to control back pain

In the above applications, as well as in many others, it is important to provide the stimulation for extended periods of time (sometimes for hours) for best results. In general the invention is most suitable for those applications wherein the stimulation can be applied without the patient being aware of it, such as while sleeping or resting. A very wide range of physical and mental ailments fall in this category.

Since prior art needed close proximity of the coils to the body a complex system of multiple coils was suggested by U.S. Pat. No. 6,658,301. The coil switching is performed by monitoring the received power in the BION devices using a communication link between the BIONs and the control. This requires the sending and receiving of commands rather than simply picking up power and commands, or even just power, from the electromagnetic field. Another problem that can be solved by the system of U.S. Pat. No. 6,658,301 is detecting that a BION is located at a point of zero mutual inductance with the transmitter coil, thus receiving no energy. Both these problems are solved by the present invention without requiring any information to be transmitted back from the receiver to the transmitter. This greatly simplifies the implanted device.

### SUMMARY OF THE INVENTION

Miniature implanted muscle, nerve and brain stimulators are powered by inductive coupling to a large coil which is preferably placed under the bed. The coil has few turns and is optimized to form part of an LC (i.e. coil and capacitor) tuned resonant circuit and generating a sufficiently large induced voltage in a small coil implanted in a person. Preferably the operation of the system is controlled by a programmable timer to operate when the user is resting or asleep. Two coils operated at two different positions can be used simultaneously to avoid spots with no signal. When the system is used to reduce angina pains, the pulsation is synchronized to the cardiac rhythm by picking up the electro-cardiac signals.

### BRIEF DESCRIPTION OF THE DRAWINGS

FIG. 1 is an isometric view of the invention with the transmitter installed under a bed.

FIG. 2 is an isometric view of the miniature implantable device.

FIG. 3 is an electrical block diagram of the miniature implantable device.

FIG. 4 is a “phantom” view of the transmitter part.

FIG. 5 is an example of the timing sequence used with the invention.

FIG. 6 is an isometric view of a transmitter using two coils to avoid loss of signal at certain positions.

FIG. 7A is a schematic view of a microwave powered implantable device.

FIG. 7B is an isometric view of a microwave powered implantable device.

FIG. 8 is an electrical block diagram of an implantable device using two coils.

FIG. 9 is a schematic view of the waveforms when using two transmitters and two frequencies to avoid loss of signal at certain positions.

FIG. 10 is a schematic view of the waveforms when using alternating transmitters to avoid loss of signal at certain positions.

### DESCRIPTION OF THE PREFERRED EMBODIMENT

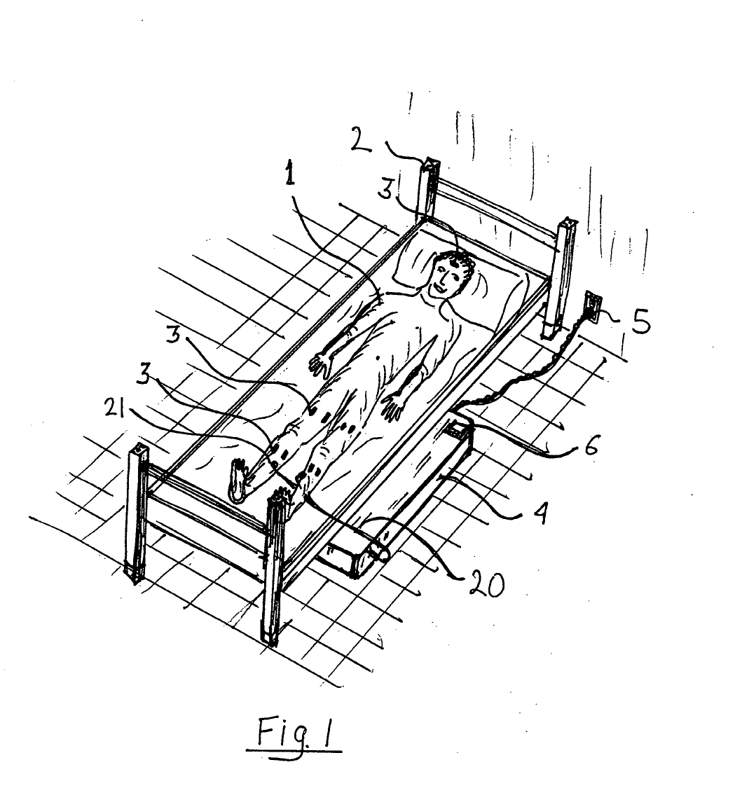

The invention comprises of two main parts: the transmitter and the implantable receiver. The receiver is similar, and sometimes can be identical, to the BION device. The BION device is described in U.S. Pat. No. 5,312,439 which is hereby incorporated by reference. As shown in FIG. 1, a patient 1 is lying on bed 2 and has implanted stimulators 3. Stimulators 3 can be muscle stimulators, brain stimulators, gastric stimulators or any other stimulator. A transmitter 4 is located under bed 1 and powered by battery power or external power, such as line power cord 5. A control panel 6 allows the selection of duration and profile of stimulation. Clearly panel 6 can be replaced by a remote control unit or external computer. For applications which need to be synchronized to a particular body rhythm, such as cardiac counter pulsation, leads 20 are attached to the body via sensing pads 21.

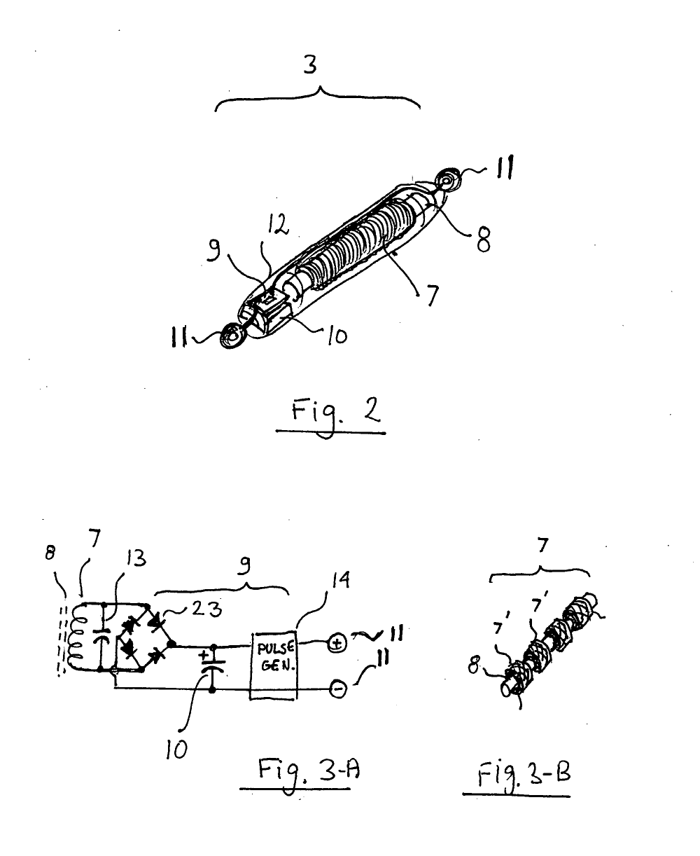

FIG. 2 shows the implantable device which could be a BION device. This device is also referred to as a “receiver” while the energy source is the “transmitter”. The implantable device 3 comprises of a coil 7 wound on a ferrite core 8, having electrodes 11 to make electrical contact inside the body. Electrodes can be an inert metal such as gold, platinum, rhodium, or tantalum. They can be metals forming a conductive oxide, such as silver or copper, or can be a combination of a metal and metal compound such as silver/silver chloride. A silicon die 9 contains a rectifier, power conditioning and a pulse generating circuit. The rectified RF signal from coil 7 is stored in capacitor 10. Silicon die 9 may contain additional circuits such as telemetry. All components except the electrodes are encased in a hermetic glass, metal or ceramic shield 12. Low melting temperature glass or a frit seal can also be used. Beneficial medicated coatings, such as drug eluting coating, or beneficial surface finishes such as sandblasting (to promote rapid bonding with tissue) can be used on the outside surfaces.

It is sometimes desirable to fill the inside of housing 10 with a polymer forming a strong bond to glass or ceramic such as silicone resin. The advantages of filling the housing are multiple: in case the glass shatters, the fragments stay bonded to the encapsulation; in case of a crack a secondary moisture barrier is formed by the encapsulation, plus the obvious ruggedness achieved by encapsulation. Some of these benefits can be achieved by an external coating; however this increases the diameter of the device.

An alternate embodiment is to build the device out of metal, with the electrodes hermetically sealed to the metal housing by glass-to-metal seals. In this type of seal the electrode lead wire is insulated with class or a ceramic material and the outside of the insulation is metalized. The metallization allows to solder or weld the electrode to the metal housing forming a hermetic seal. Such seals are common in implantable medical devices such as pacemakers. Surprisingly, the attenuation of such a metal enclosure to the RF frequency used can be minimal (under 10%) if sufficiently thin metal of low conductivity and, optionally, high permeability is used. By the way of example, a receiver having a diameter of 2 mm housed in a metal tube made of 50 um thick ferromagnetic stainless steel and glass-to-metal electrode seals had practically identical signal strength when tested between 500 KHz to 1 MHz with and without the metal enclosure. This surprising result is partially explained by the “skin depth” of RF penetration, which goes down as the square root of the permeability and conductivity. The metal enclosure represents a high impedance compared to the copper in the coil, therefore not having much effect on the receiver signal. Filling the inside of the tube with rigid electrical insulation material such as epoxy resin allows the use of very thin walls, further improving performance.

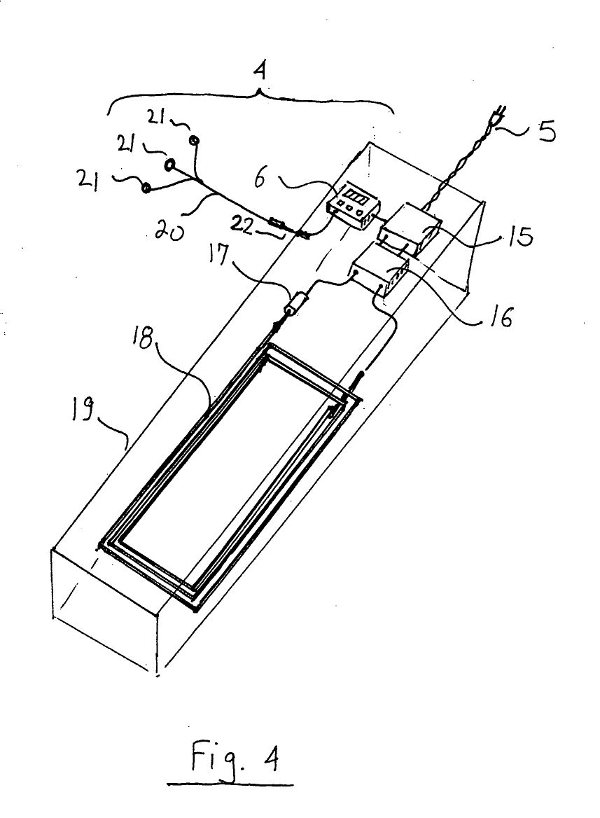

FIG. 3A shows the electrical circuit of the implant. Capacitor 13 is optional and is used to form a resonant circuit together with coil 7, in order to gain some power. The gain depends on how heavily the coil is loaded. Rectifier 23 can be of the Schottky type. Storage capacitor 10 is a solid tantalum type, having a typical value of a few uF. Normally it only needs to store energy for less than 100 mS, as the lowest output pulse frequency is normally above 10 Hz. The pulse generator is conventional and the output pulse is typically about 10V with a current of 1 mA and a duration of 100 uS-500 uS. Typical pulsing frequency is 20 Hz to 50 Hz for muscle stimulation. Even when pulsed at a relatively high rate of 100 Hz the average power is 10V×1 mA×0.1 mS/10 mS=100 uW. Obviously the capacitor can be replaced by a rechargeable battery or a supercapacitor. Unlike the original BION, the coil is wound in a manner maximizing power output by minimizing internal capacitance. This is shown in FIG. 3B. High internal capacitance will cause self-resonance at frequencies near the operating frequency. Coil 7 is divided into segments 7′ which are in series. Each segment can have a regular winding or a skew winding, similar to RF inductors. The skew winding minimizes capacitance between turns but takes more space. By the way of example, the coil is made up of 4 segments with about 1000 turns of 25 um wire each. The self resonance was found to be at 2 MHz and the operating frequency was 1 MHz. The details of such coils as well as integrating the components onto a silicon die are well known in the art of electrical engineering. FIG. 4 shows the details of a basic transmitter 4. A digital switching power supply 15 is powered by line power via cord 5. If needed, portable battery power can be used. The switching supply generated a sine wave output at a frequency typically between 200 KHz and 10 MHz. A narrowband filter 16 further filters the output to a pure sine wave. The last stage of filtering is provided by using a resonant circuit formed by transmitter coil 18 and capacitor 17. The need for careful filtering of the sine wave is driven by the need to minimize RFI (Radio Frequency Interference) to adjacent devices. By the way of example, generator 15 is a FET based sine wave generator operating at 1 MHz. Such generators are commercially available as they are widely used for induction heating and other industrial application. Filter 16 is a passive filter tuned to 1 MHz and having a bandwidth of below 1 KHZ. Coil 18 is made of 5 turns of copper ribbon and has a rectangular (or oval) shape of about 50 cm×90 cm. The air gap between turns is about 15 mm. The copper ribbon is about 1 mm thick and 20 mm wide. The inductance is about 50 uH and the resonating capacitor 17 is about 500 pF. Since the required magnetic field strength above the bed is about 0.1 mT, the coil has to be driven at a current of about 40 A. The voltage across the coil is significant: V=2.Pi.f.L.I=2×3.14×10⁶×50×10⁻⁶×40=about 12.5 KV. In this equation L=inductance, I=current, f=frequency. The coil has to be well insulated inside non-conductive box 19. One possible construction is to fill the whole volume of box 19 with a low density foam such as Styrofoam. The drive voltage required for the tuned circuit is Q times lower than the coil voltage, thus for a tuned circuit with a Q (Q is the “quality factor”) of about 500 the actual drive voltage is only 25V. The power dissipated on the resistive component, based on the above dimensions, is 40 A²×0.02=32 W.

While the example uses 1 MHz operating frequency, the exact operating frequency is not critical and should be selected based on regulatory frequency allocations, the desire to minimize interference with other electronic devices and tissue absorption.

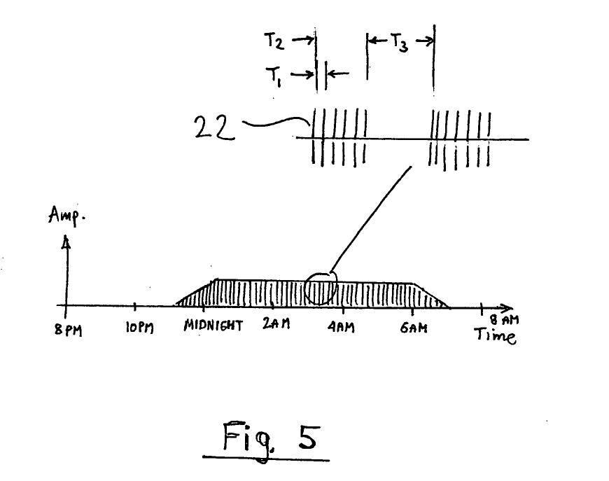

Control unit 16, which could also be a remote control unit, allows to program the start and stop times as well as intensities of the stimulation. Some applications, such as muscular counter pulsation, require a synchronization signal from the patient such as EKG. Such synchronization can be picked up by a wireless link or by a detachable wire 20 having a connector 22 and electrodes 21, as shown in FIG. 1. The electrodes are attached to the proper points in the body to pick up the desired synchronization signal. FIG. 5 shows a typical timing sequence. Stimulation pulses 22 start after patient is asleep (around midnight in the example of FIG. 5) and stop before patient wakes up (around 6 am in this example). The intensity of the stimulation is increased gradually when stimulation starts and is decreased gradually when stimulation ends in order to minimize sudden changes during sleep. The intensity of the stimulation is changed by changing the transmitted power from the transmitter coil or by having a more complex receiver capable of receiving commands and storing parameters.

It is well known that the coupling between coils can drop to zero when the axis of the receiver coil is perpendicular to the magnetic field lines of the transmitter coil. Since the position of the patient may change during sleep it may be desirable to offer a system in which power is always available in receiver regardless of position. This is achieved by using two or more coils as shown in FIG. 6. Coil 18 is mounted at an angle θ to coil 18′. The coils can be combined as one structure or physically separate. When the axis of the receiver coil is perpendicular to the magnetic field from coil 18 it is not perpendicular to the field from coil 18′, therefore a signal from one of the coils is always present. Capacitors 17 and 17′ tune each circuit to different frequency. Both frequencies are picked up by the receiver and rectified, but they can not be zero at the same time. The difference in frequency is needed to avoid destructive interference in the receiver coil. The difference can be as small as 100 Hz and as large as 1 MHz. When a large difference is used, it is best not to use a tuned receiver. In a different embodiment, both coils can be fed by the same frequency but alternate, the energy storage capacitor 10 in FIG. 3A storing sufficient energy for the periods that one of the coils does not induce a sufficient voltage in the receiver. In case of alternating coils it is desired to increase and decrease the power gradually in each coil, in order to minimize the creation of Electro Magnetic Interference (EMI). More details about this method are discussed later.

In theory three coils are needed to guarantee coupling under all conditions, as the receiver can be perpendicular to the field from both coils. In practice some positions of the patient on the bed are unlikely (such as sleeping standing up), thus two coils are sufficient if their orientation is correctly chosen.

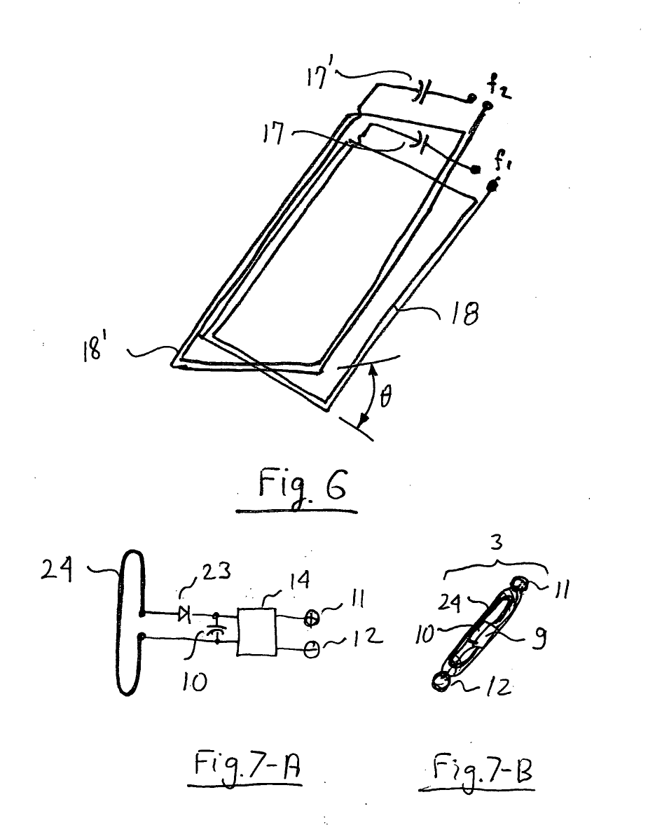

FIGS. 7A and 7B show an alternative method of coupling energy to the receiver. Instead of using “transformer action”, a true transmitter and receiver is used. Such a system operates at a significantly higher frequency, typically 100 MHz to 10 GHz. The receiver can be a folded dipole or similar antenna 24 rectified by rectifier 23, and activating pulse generator 14 via energy storage capacitor 10. The outside appearance of such a receiver is shown in FIG. 7B. One advantage of such a system is that the transmitter can be made directional by using a directional antenna. When using a directional transmitter, a similar problem of the received signal dropping to zero at certain orientations exists. This can be solved in a similar manner described for induction coils, by using two or more transmitting antennae with different frequencies or using a single frequency and alternating the active antenna at a rapid rate. The same EMI considerations as apply to coils apply to antennae. When using very high frequencies two different polarizations can also be used in the transmitted signal to avoid a “null” in the reception.

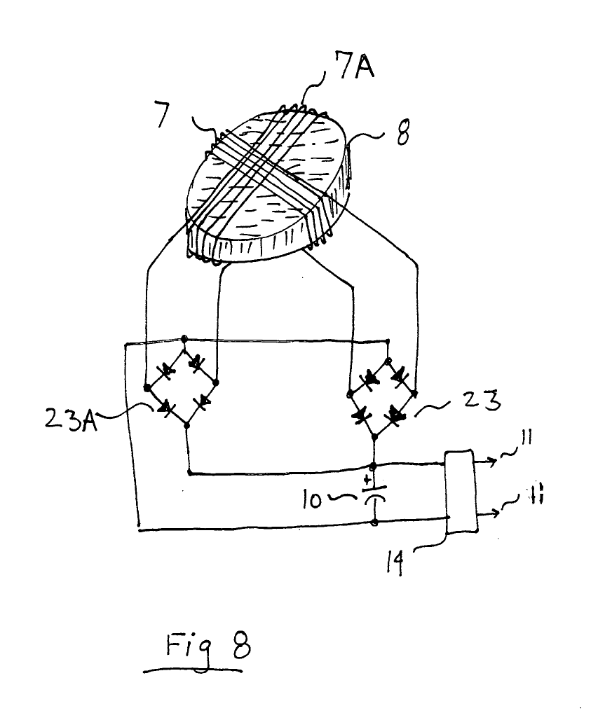

An alternate embodiment for avoiding reception nulls is shown in FIG. 8. A single transmitter coil is used at a single frequency and the receiver has at least two coils wound is different directions, to avoid having a null in both coils at the same spatial position. The output from the coils can not be simply added, as the outputs can cancel each other and form a new null. This could happen if the outputs were equal but having opposite phases. If the outputs are rectified first, the DC voltages can be added together as they always have the same polarity. Coils 7A and 7B are wound on a ferrite 8. The output of the coils is rectified by rectifiers 23 and 23A and fed to a capacitor 10 which powers pulse generator 14. It may appear that only the coil with the higher output will charge the capacitor, however because of the large output impedance of the coils, the voltage of this coil will drop and both coils will share the charging of the capacitor. Other modes of adding together the DC output of the coils, such as connecting the DC voltages in series, can also be used. As before, two coils are sufficient for most application and theoretically three coils will cover all spatial positions. Placing a second coil in the receiver increases the receiver size slightly but greatly simplifies the transmitter. The same considerations apply to any “far field” coupling schemes using higher RF frequencies: a receiver can have two antennae oriented at different angles or polarizations.

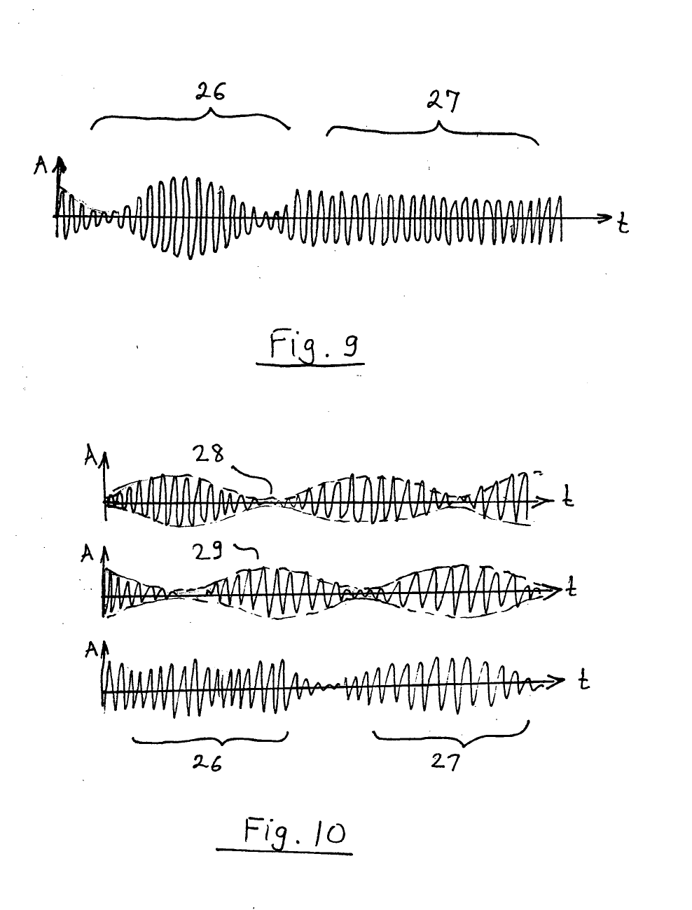

FIG. 9 shows typical waveforms when using multiple transmitters and multiple frequencies to avoid “nulls” or points of no received signal. FIG. 10 shows the method using multiple transmitters using a single frequency. In this figure the amplitude of each transmitter is modulated sinusoidally, to minimize EMI, but any other modulation envelope can be used. Both FIG. 9 and FIG. 10 show the output from the receiver coil before rectification and filtering by the storage capacitor (capacitor 10 in FIG. 3A). Clearly the amplitude variations shown will be smoothed out by the storage capacitor as long as there is no extended period of low signal. The time constant of the storage capacitor has to be larger than the longest period of low signal. FIG. 9 shows the use of two frequencies. When using two frequencies a beat is formed between them, as shown in part 26 of FIG. 9. The frequency of the beat is the difference between the frequencies. Since the storage capacitor is typically small (a few uF) the beat needs to be sufficiently rapid, for example 100 Hz to a few KHz. Too large a separation between the transmitted frequencies wastes bandwidth. Part 27 of both figures shows the signal when the receiver can only pick up signal from one transmitter. FIG. 10 shows the use of a single frequency and alternating coils or transmitter antennae. Graph 28 is the output of the first transmitter, graph 29 is the output of the second. Graph 26 is a typical signal when both transmitters can be picked up, in 27 only one is picked up. The ability to have signal at all position is important for allowing the patient freedom of movement, both while asleep and awake.

An important application for the present invention is the reduction of angina in cardiac patients by using muscular counter pulsation. By stimulating the leg muscle from the distal to proximal part, a peristaltic pump is formed by the blood vessels which acts in a similar manner to a balloon pump to offload the heart. It was found that just a few hours of this treatment per week can reduce or eliminate angina for periods of weeks to months. A survey article on the benefits of this method was published in J. of American College of Cardiology Vol. 33 No. 7 1999, pp. 1833-1840 and is hereby incorporated by reference. While the tests reported in this article were done by pneumatic cuffs rather than muscle stimulation, it is reported that muscle stimulation has a similar effect when used for counter pulsation. For this application it is best to synchronize the pulsation with the EKG waveform picked up by leads 20 and pads 21 in FIG. 1.

While the main examples in the disclosure use a transmitter located under a bed, it is obvious that other suitable locations can be used such as under (or behind) a chair and on a desk.

More surprisingly, when such stimulators were placed in the brain tests proved them effective against severe depression, including the type resistant to medication. Other tests reported in medical journals showed benefit for treating epilepsy, alcoholism, and migraine headaches. In each one of these cases the placement of the stimulators is important and more data is emerging. Battery powered brain stimulators have been used for a long time but their bulk was prohibitive. Wireless stimulators open many new possibilities.

What is claimed is:

1. A system for the wireless stimulation of parts of a human body, comprising:
at least one transmitter not attached to the body and at least one receiver implanted in the body, said receiver having at least two coils wound at different orientations.

2. A system as in claim 1 wherein said transmitter is located under a bed and is capable of turning on and off automatically.

3. A system as in claim 1 wherein said transmitter is located under a chair.

4. A system as in claim 1 wherein said transmitter is located on a desk.

5. A system as in claim 1 wherein the output of said receiver coils is rectified and the DC power produced is added together.

6. A system as in claim 1 wherein the body is in a state of sleep.

7. A system as in claim 1 wherein the receiver is housed inside a metal enclosure filled with a rigid electrical insulator.

8. A system for the wireless stimulation of parts of a human body having at least one implantable stimulator having a receiver coil comprising of separate segments connected in series.

9. A system for assisting the operation of a human heart by contracting muscles outside the heart in synchronism with the heart, said system containing a plurality of implanted receivers having a wireless coupling to a transmitter.

10. A system as in claim 9 wherein said synchronism is performed by the detecting the electro-cardiac activity in said body.

11. A system as in claim 9 wherein said muscles are located in the legs and operate in a sequence causing a blood counter pulsation action.

12. A system as in claim 9 wherein said coupling is operational regardless of the relative position between the said transmitter and receivers.

13. A system as in claim 9 wherein said assisting is being performed while the patient is asleep.

14. A system as in claim 1 used for weight loss.

15. A system as in claim 1 used for strengthening muscles.

16. A system as in claim 1 used for sleep apnea.
